SEQUENCE LISTING (56 sequences)

SEQ ID NO: 1 - DNA, 18 bp, Artificial
  Antisense Oligonucleotide
ggcttctcctccatggtc

SEQ ID NO: 2 - DNA, 20 bp, Artificial
  Antisense Oligonucleotide
cggcttctcctccatggtcc

SEQ ID NO: 3 - DNA, 20 bp, Artificial
  Antisense Oligonucleotide
gggcttctcctccaatgtcc

SEQ ID NO: 4 - DNA, 20 bp, Artificial
  Antisense Oligonucleotide
tcttccgcgacttgctccgc

SEQ ID NO: 5 - DNA, 20 bp, Artificial
  Antisense Oligonucleotide
ctgttcaccgtgcaccctgt

SEQ ID NO: 6 - DNA, 18 bp, Artificial
  Antisense Oligonucleotide
ggtcttctcacacatcgg

SEQ ID NO: 7 - DNA, 20 bp, Artificial
  Antisense Oligonucleotide
tggtcttctcacacatcggc

SEQ ID NO: 8 - DNA, 20 bp, Artificial
  Antisense Oligonucleotide
tggtcttctcacacatgggc

SEQ ID NO: 9 - DNA, 20 bp, Artificial
  Antisense Oligonucleotide
cctgtacgcgttgatctcca

SEQ ID NO: 10 - DNA, 20 bp, Artificial
  Antisense Oligonucleotide
agtcgcctagtcgagtcctt

SEQ ID NO: 11 - DNA, 20 bp, Artificial
  Antisense Oligonucleotide
attgttgatggtggtcttct

SEQ ID NO: 12 - DNA, 20 bp, Artificial
  Antisense Oligonucleotide
tcatgcttgttgatgtggtt

SEQ ID NO: 13 - DNA, 20 bp, Artificial
  Antisense Oligonucleotide
gcggtagttgtactcattgt

SEQ ID NO: 14 - DNA, 20 bp, Artificial
  Antisense Oligonucleotide
tgtcgttcgagttgatcgta

SEQ ID NO: 15 - DNA, 20 bp, Artificial
  Antisense Oligonucleotide
gtggtccagcagcggtagtt

SEQ ID NO: 16 - DNA, 20 bp, Artificial
  Antisense Oligonucleotide
gacgttagcgtgcgatgtgc

SEQ ID NO: 17 - DNA, 18 bp, Artificial
  Antisense Oligonucleotide
cttattccccacaatgta

SEQ ID NO: 18 - DNA, 18 bp, Artificial
  Antisense Oligonucleotide
cttgttccccacaatgta

SEQ ID NO: 19 - DNA, 18 bp, Artificial
  Antisense Oligonucleotide
catagcacattcttctca

SEQ ID NO: 20 - DNA, 18 bp, Artificial
  Antisense Oligonucleotide
accgttcatcagttcact

SEQ ID NO: 21 - DNA, 18 bp, Artificial
  Antisense Oligonucleotide
tcctccggagccagactt

SEQ ID NO: 22 - DNA, 18 bp, Artificial
  Antisense Oligonucleotide
cagctactcgcatgctgc

SEQ ID NO: 23 - DNA, 21 bp, Artificial
  siRNA reagent
  misc_feature (20)..(21): wherein “n” represents “t” thymine
  this is a combined DNA/RNA sequence
gccgaugugugagaagaccnn

SEQ ID NO: 24 - DNA, 18 bp, Artificial
  siRNA reagent
  misc_feature (17)..(18): wherein “n” represents “t” thymine
  this is a combined DNA/RNA sequence
gccgaugugugagaagnn

SEQ ID NO: 25 - DNA, 24 bp, Artificial
  siRNA reagent
  misc_feature (23)..(24): wherein “n” represents “t” thymine
  this is a combined DNA/RNA sequence
gccgaugugugagaagaccaccnn

SEQ ID NO: 26 - DNA, 27 bp, Artificial
  siRNA reagent
  misc_feature (26)..(27): wherein “n” is “t” thymine
  this is a combined DNA/RNA sequence
gccgaugugugagaagaccaccaucnn

SEQ ID NO: 27 - DNA, 21 bp, Artificial
  siRNA reagent
  misc_feature (20)..(21): wherein “n” represents “t” thymine
  this is a combined DNA/RNA sequence
ccagaagaguguguagccgnn

SEQ ID NO: 28 - DNA, 21 bp, Artificial
  wherein “n” represents “t” thymine
  this is a combined DNA/RNA sequence
  misc_feature (20)..(21): wherein “n” represents “t” thymine
gccgaugugugugaagaccnn

SEQ ID NO: 29 - DNA, 21 bp, Artificial
  siRNA reagent
  misc_feature (20)..(21): wherein “n” represents “t” thymine
  this is a combined DNA/RNA sequence
gcccaugugugagaagaccnn

SEQ ID NO: 30 - DNA, 21 bp, Artificial
  siRNA reagent
  misc_feature (20)..(21): wherein “n” is “t” thymine
  this is a combined DNA/RNA sequence
cuacauuguggggaauaagnn

SEQ ID NO: 31 - DNA, 21 bp, Artificial
  siRNA reagent
  misc_feature (20)..(21): wherein “n” is “t” thymine
  this is a combined DNA/RNA sequence
gaauaagggguguuacaucnn

SEQ ID NO: 32 - DNA, 21 bp, Artificial
  siRNA reagent
  misc_feature (20)..(21): wherein “n” is “t” thymine
  this is a combined DNA/RNA sequence
cuacauuguggggaacaagnn

SEQ ID NO: 33 - DNA, 21 bp, Artificial
  siRNA reagent
  misc_feature (20)..(21): wherein “n” is “t” thymine
  this is a combined DNA/RNA sequence
ggucuucucacacaucggcnn

SEQ ID NO: 34 - DNA, 18 bp, Artificial
  siRNA reagent
  misc_feature (17)..(18): wherein “n” is “t” thymine
  this is a combined DNA/RNA sequence
cuucucacacaucggcnn

SEQ ID NO: 35 - DNA, 24 bp, Artificial
  siRNA reagent
  misc_feature (23)..(24): wherein “n” is “t” thymine
  this is a combined DNA/RNA sequence
gguggucuucucacacaucggcnn

SEQ ID NO: 36 - DNA, 27 bp, Artificial
  siRNA reagent
  misc_feature (26)..(27): wherein “n” is “t” thymine
  this is a combined DNA/RNA sequence
gaugguggucuucucacacaucggcnn

SEQ ID NO: 37 - DNA, 21 bp, Artificial
  siRNA reagent
  misc_feature (20)..(21): wherein “n” is “t” thymine
  this is a combined DNA/RNA sequence
cggcuacacacucuucuggnn

SEQ ID NO: 38 - DNA, 21 bp, Artificial
  siRNA reagent
  misc_feature (20)..(21): wherein “n” is “t” thymine
  this is a combined DNA/RNA sequence
ggucuucacacacaucggcnn

SEQ ID NO: 39 - DNA, 21 bp, Artificial
  siRNA reagent
  misc_feature (20)..(21): wherein “n” is “t” thymine
  this is a combined DNA/RNA sequence
ggucuucucacacaugggcnn

SEQ ID NO: 40 - DNA, 21 bp, Artificial
  siRNA reagent
  misc_feature (20)..(21): wherein “n” is “t” thymine
  this is a combined DNA/RNA sequence
cuuauuccccacaauguagnn

SEQ ID NO: 41 - DNA, 21 bp, Artificial
  siRNA reagent
  misc_feature (20)..(21): wherein “n” is “t” thymine
  this is a combined DNA/RNA sequence
gauguaacaccccuuauucnn

SEQ ID NO: 42 - DNA, 21 bp, Artificial
  siRNA reagent
  misc_feature (20)..(21): wherein “n” is “t” thymine
  this is a combined DNA/RNA sequence
cuuguuccccacaauguagnn

SEQ ID NO: 43 - DNA, 15 bp, Artificial
  Antisense Oligonucleotide
attgtggggaataag

SEQ ID NO: 44 - DNA, 15 bp, Artificial
  Antisense Oligonucleotide
catggaggagaagcc

SEQ ID NO: 45 - DNA, 15 bp, Artificial
  Antisense Oligonucleotide
atgtgtgagaagacc

SEQ ID NO: 46 - DNA, 15 bp, Artificial
  Antisense Oligonucleotide
agaccaccatcaaca

SEQ ID NO: 47 - DNA, 15 bp, Artificial
  Antisense Oligonucleotide
gagtacaactaccgc

SEQ ID NO: 48 - DNA, 15 bp, Artificial
  Antisense Oligonucleotide
ccgctgctggaccac

SEQ ID NO: 49 - DNA, 150 bp, Homo sapiens
tgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaa

SEQ ID NO: 50 - DNA, 18 bp, Artificial
  Antisense Oligonucleotide
tacattgtggggaataag

SEQ ID NO: 51 - DNA, 18 bp, Artificial
  Antisense Oligonucleotide
gaccatggaggagaagcc

SEQ ID NO: 52 - DNA, 18 bp, Artificial
  Antisense Oligonucleotide
ccgatgtgtgagaagacc

SEQ ID NO: 53 - DNA, 18 bp, Artificial
  Antisense Oligonucleotide
agaagaccaccatcaaca

SEQ ID NO: 54 - DNA, 18 bp, Artificial
  Antisense Oligonucleotide
aatgagtacaactaccgc

SEQ ID NO: 55 - DNA, 18 bp, Artificial
  Antisense Oligonucleotide
ctaccgctgctggaccac

SEQ ID NO: 56 - DNA, 1000 bp, Homo sapiens
ttttttttttttttgagaaagggaatttcatcccaaataaaaggaatgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcat

(19) United States
(12) Patent Application Publication
(10) Pub. No.: US 2007/0213284 A1
(43) Pub. Date: Sep. 13, 2007

(54) Method of selecting targets for gene silencing by RNA interference

(76) Inventors: Muhammad Sohail, Oxford (GB); Valentine Moya Macaulay, Oxford (GB)

Correspondence Address: FOLEY & LARDNER LLP, 111 HUNTINGTON AVENUE, 26TH FLOOR, BOSTON, MA, 02199-7610, US

(21) Appl. No.: 10/996,865

(22) Filed: Nov. 24, 2004

Related U.S. Application Data
(63) Continuation of application No. PCT/GB03/02307, filed on May 28, 2003.

(30) Foreign Application Priority Data
May 28, 2002 (GB) 0212302.4

(51) Int. Cl. A61K 31/7105; A61K 31/713; C07H 21/02; C12N 15/64

(52) U.S. Cl. 514/44; 435/91.4; 536/24.5

(57) ABSTRACT

The invention relates to the selection of target regions of RNA transcripts for specific down-regulation of gene expression by RNA interference. Target regions of the transcript are identified on the basis of hybridisation to scanning arrays of antisense oligonucleotides and siRNA reagents comprising double-stranded RNAs corresponding to the target regions are then synthesised.

### FIELD OF THE INVENTION

The invention relates to the preparation of double-stranded RNA reagents for use in the specific down-regulation of gene expression by RNA interference by hybridisation to scanning arrays of antisense oligonucleotides.

### BACKGROUND TO THE INVENTION

RNA interference (RNAi) is a process of sequence-specific gene silencing initiated by double-stranded RNA (dsRNA) that is homologous in sequence to a region of the silenced gene (Fire, A. Trends Genet. Vol. 15, 358-363, 1999; Sharp, P. A. Genes Dev. Vol. 15, 485-490, 2001).

Elbashir et al. (Nature, 411, 494-498, 2001) have demonstrated effective RNAi-mediated gene silencing in mammalian cells using dsRNA fragments of 21 nucleotides in length (also termed small interfering RNAs or siRNAs). These short siRNAs demonstrate effective and specific gene silencing, whilst avoiding the interferon-mediated non-specific reduction in gene expression which has been observed with the use of dsRNAs greater than 30 bp in length (Stark G. R. et al., Ann Rev Biochem. 1998, 67: 227-264; Manche, L et al., Mol Cell Biol., 1992, 12: 5238-5248). Thus, siRNAs represent promising candidate gene-specific therapeutic agents, providing an alternative to anti-sense oligonucleotides.

One potential problem to be overcome in the development of siRNA agents for any given gene is the selection of an appropriate region of the gene to target in order to achieve effective gene-silencing by RNAi.

In the field of anti-sense oligonucleotide technology suitable single-stranded antisense oligonucleotides for a given gene may be selected with the use of scanning arrays (Southern, E. M. et al., Nucleic Acids Res., 1994, 22(8): 1368-1373; Sohail, M. and Southern, E. M. “Using oligonucleotide scanning arrays to find effective antisense reagents”, Methods in Molecular Biology, vol. 170: DNA Arrays: Methods and Protocols, Ed J. B. Rampal, Humana Press Inc., Totowa, N.J.; Sohail, M. et al., Nucleic Acids Res., 2001, 29(10): 2041-2051).

The present inventors have now shown that the sequences selected for use in single-stranded antisense oligonucleotides on basis of array screening can also be used as the basis of siRNA reagents that are effective in gene silencing by RNAi.

### DESCRIPTION OF THE INVENTION

In accordance with a first aspect of the invention there is provided a method of preparing an siRNA reagent for use in gene silencing of a target gene by RNA interference, which method comprises:

(a) preparing a scanning array of antisense oligonucleotides spanning a region of a transcript of the target gene;

(b) hybridising to the array labelled transcripts of the target gene;

(c) identifying an oligonucleotide within the array which hybridizes with the labelled transcripts; and

(d) preparing an siRNA reagent comprising a double-stranded RNA of identical sequence to the oligonucleotide identified in step (c).

As aforesaid, it is known to use scanning arrays of antisense oligonucleotides in order to identify regions of a particular transcript to target for inhibition of gene expression using antisense oligonucleotides. However, it was surprising to observe that sequences identified using the scanning array as being useful as antisense oligonucleotides may also mediate gene silencing by RNA interference because the mechanisms of action of antisense oligonucleotides and RNA interference are very different (see review by Brantl, S. Antisense-RNA regulation and RNA interference, Biochem Biophys Acta. Vol. 1575(1-3), 15-25, 2002).

The term “siRNA reagent” refers to a nucleic acid molecule that is capable of down-regulating expression of a target gene by RNA interference. The characteristics of siRNA reagents are generally known in the art. siRNA reagents generally comprise a region of double-stranded RNA, although as discussed below one or more bases in the double-stranded RNA may be replaced with DNA bases. The double-stranded RNA may be flanked by short single-stranded overhangs, as described below.

Scanning oligonucleotide arrays comprising oligonucleotides complementary to a target region of a mRNA transcript of the gene of interest may be synthesised using the methods which are known in the art and described, for example by Southern E. M. et al., Nucleic Acids Res., 1994, 22(8): 1368-1373 and Sohail, M. and Southern, E. M. “Using oligonucleotide scanning arrays to find effective antisense reagents”, Methods in Molecular Biology, vol. 170: DNA Arrays: Methods and Protocols, Ed J. B. Rampal, Humana Press Inc., Totowa, N.J., the contents of which documents are incorporated herein by reference. A detailed protocol for synthesis of scanning arrays is also given in the accompanying examples.

Scanning arrays are a simple tool that allow combinatorial synthesis of a large number of oligonucleotides on a solid platform (typically glass or polypropylene, see note 1 in accompanying examples) in a spatially addressable fashion, and parallel measurement of the binding of all oligonucleotides complementary to the target mRNA.

The scanning arrays comprise sets of oligonucleotides of various lengths. A series of oligonucleotides, complementary to the target mRNA, is made by sequential coupling of nucleotides to a solid surface. The DNA synthesis reagents are applied to a confined area on the surface of the solid support using a mask (see below). The mask is shifted along the surface after each round of coupling, resulting in a series of oligonucleotides each complementary to a region of the target sequence.

The “gene of interest” may be essentially any gene for which it is desired to develop an siRNA/RNAi reagent. The method of the invention is of general utility, thus the precise nature of this gene (and the mRNAs transcribed therefrom) is not material to the invention. The “target region” may be a sub-fragment of the gene of interest which it is desired to test in order to identify region(s) which may potentially be useful targets for gene silencing by RNA interference. The “target region” may be an arbitrarily chosen sub-fragment of the gene of interest or may have been selected on the basis of an assay for potentially suitability as an RNAi target. For example, the “target region” may be one which is relatively accessible in the mRNA transcript because of a relative lack of secondary structure. Regions of mRNA transcripts which are potentially accessible for gene silencing may be identified by RNaseH mapping (see Sohail et al., Nucleic Acids Res., 2001, 29(10): 2041-2051).

The scanning arrays will generally containing all complements of the selected target sequence up to a maximum length determined by the size of the template and template displacement used in the synthesis of the scanning array (Southern et al. 1994, ibid). Typically, the maximum length of the oligonucleotides in the array will be around 18-20 nt, but this may be varied if required. The short dsRNAs (siRNAs) used for RNA interference are typically 21-23 bp in length, hence it would be appropriate to include similar length sequences in the array.

The scanning arrays are hybridised with a probe which is a transcript of the gene of interest labelled with a revealing label, which may be essentially any type of revealing label which permits visualisation and quantitation of the hybridisation intensity. Radiolabels are particularly preferred. Suitable labelled RNA probes may be conveniently synthesised using standard techniques known in the art (see accompanying examples).

Typically the hybridisation will be carried out at a temperature in the range 20-37° C. Hybridisation at 37° C. is particularly preferred, since oligonucleotides which hybridise at this temperature are more likely to be effective in vivo. The compositions of standard hybridisation buffers which are preferred for use with the arrays are given in the accompanying examples.

Oligonucleotides which hybridise to the array are identified and double-stranded RNA reagents (also referred to herein as RNAi reagents or siRNAs) having identical sequence are synthesised. The inventors have demonstrated by experiment that hybridisation to the scanning array is directly predictive of effectiveness in RNA interference. In this context “identical sequence” is not intended to be interpreted literally as requiring 100% sequence identity. The sequence of the dsRNA may differ slightly from the antisense oligonucleotide (ASO) sequence. For example the length of the dsRNA may be longer or shorter by several nt to optimise performance of the dsRNA. The RNA duplex will preferably be less than 30 bp in length, since duplexes of greater than 30 bp may induce non-specific interferon-mediated effects when introduced into cells in vivo. RNA duplexes of 20-27 bp, and typically 20-24 bp, in length are particularly suitable as RNAi reagents. If the dsRNA is made longer than the ASO then the extra sequence may correspond to the “native” sequence of the mRNA. The dsRNA may contain one or more substitute bases in order to optimise performance in RNAi. Substitution of even a single nucleotide may have a profound effect on activity of the RNAi duplex. The dsRNA may further contain non-natural bases or non-natural backbone linkages, for example to enhance stability in vivo or enhance resistance to degradation by nucleases. The dsRNA may also include single-stranded overhangs at one or both ends of the duplex. In a particularly preferred embodiment the dsRNA may contain 3′ overhanging nucleotides, preferably 3′ overhanging thymidines (dTdT) or uridines (UU).

siRNA reagents may be formed of RNA/DNA chimeras. These chimeras include, for example, the siRNA reagents comprising a double-stranded RNA with 3′ overhangs of DNA bases (e.g. dTdT), as discussed above, and also siRNA reagents comprising a double-stranded “RNA” in which one or more of the RNA bases, or even an entire strand, are replaced with DNA bases.

In a further embodiment rather than being formed of two separate RNA strands annealed together, the si RNA reagent may comprise a dsRNA having a foldback stem-loop or hairpin structure, wherein the two strands of the dsRNA are covalently linked. RNAs having this structure are typical if the dsRNA is synthesised by expression in vivo or by in vitro transcription. The precise nature and sequence of the “loop” linking the two RNA strands is generally not material to the invention, except that it should not impair the ability of the double-stranded part of the molecule to mediate RNAi.

The double-stranded RNA will preferably comprise 20-27, or 20-24, consecutive nucleotides of the target mRNA sequence, since duplexes of this length are particularly effective in RNAi.

Double-stranded RNAs may be synthesised in vitro using chemical or enzymatic RNA synthesis techniques well known in the art. In one approach the two separate RNA strands may be synthesised separately and then annealed to form double-strands.

In a further embodiment, double-stranded RNAs may be synthesised by intracellular expression from a suitable expression vector. Thus, the invention further provides a method of preparing an expression vector capable of expressing an siRNA reagent for use in gene silencing of a target gene by RNA interference, which method comprises:

(a) preparing a scanning array of antisense oligonucleotides spanning a region of a transcript of the target gene;

(b) hybridising to the array labelled transcripts of the target gene;

(c) identifying an oligonucleotide within the array which hybridizes with the labelled transcripts; and

(d) preparing an expression vector capable of expressing an siRNA reagent comprising a double-stranded RNA of identical sequence to the oligonucleotide identified in step (c).

A number of expression vector systems for in vivo expression of short double-stranded RNAs for use as RNAi reagents (also referred to as small interfering RNAs, or siRNAs) are known in the art. Generally, siRNAs are expressed as stem-loops, which may be rapidly processed within the cell to produce the “free” siRNA (see review by Tuschl, Nature Biotechnology, Vol. 20(5), 446-448, 2002). Vector systems for expression of siRNAs are often based on RNA Pol III promoters, since these are particularly suited to accurate expression of very short RNA sequences. Suitable vector systems are described in Brummelkamp, T. R. et al., Science, Vol. 296, 550-553, 2002; Lee, N. S. et al., Nature Biotechnology, Vol. 20, 500-505, 2002; Miyagashi, M & Taira, K. Nature Biotechnology, Vol. 20, 497-500, 2002; Paul, C. P. et al., Nature Biotechnology, Vol. 20, 505-508, 2002, the contents of which are incorporated herein by reference.

In a further aspect the invention provides a method of preparing a pharmaceutical composition comprising an siRNA reagent capable of mediating gene silencing of a target gene by RNA interference, which method comprises:

preparing an siRNA reagent capable of mediating gene silencing of a target gene by RNA interference according to the method described above and formulating the siRNA reagent into a pharmaceutical composition comprising the siRNA reagent and one or more diluents, excipients or carriers.

siRNA reagents may be formulated into pharmaceutical compositions comprising a therapeutically effective amount of the siRNA nucleic acid in combination with any standard physiologically and/or pharmaceutically acceptable carriers known in the art. “Pharmaceutically acceptable” means a non-toxic material which does not interfere with the activity of the pharmaceutically active ingredients in the composition. “Physiologically acceptable” refers to a non-toxic material that is compatible with a biological system such as a cell, tissue or organism. Physiologically and pharmaceutically acceptable carriers may include diluents, fillers, salts, buffers, stabilizers, solubilizers etc.

For delivery into cells in vivo siRNAs may be formulated with lipid-based carriers including, for example, oil-in water emulsions, micelles, and liposomes. Liposomes are the most preferred carriers, and there use is well known in the art. Liposomes are commercially available from Gibco BRL, for example, as LIPOFECTIN™ and OLIGOFECTAMINE™, which are formed of cationic lipids. Methods for making liposomes are well known in the art and have been described in many publications. Liposomes may be targeted to a particular tissue by coupling the liposome to a particular tissue by coupling the liposome to a specific ligand, such as a monoclonal antibody, sugar, glycolipid or protein.

Liposomes may also be used to deliver vectors encoding siRNAs. In the field of human gene therapy it is well known to deliver expression vectors, such as plasmids, via nucleic acid-liposome complexes.

For use in human therapy, pharmaceutical compositions including the siRNAs of the invention will be administered to a patient in need of treatment in a “therapeutically acceptable amount”. A therapeutically acceptable amount is an amount of a pharmaceutical preparation that alone, or together with further doses, produces the desired response in the condition being treated. The precise amount of the composition administered will, however, generally be determined by a medical practitioner, based on the circumstances pertaining to the disorder to be treated, such as the severity of the symptoms, the composition to be administered, the age, weight, and response of the individual patient and the chosen route of administration.

The invention will be further understood with reference to the following experimental protocols, together with the accompanying Figures, in which:

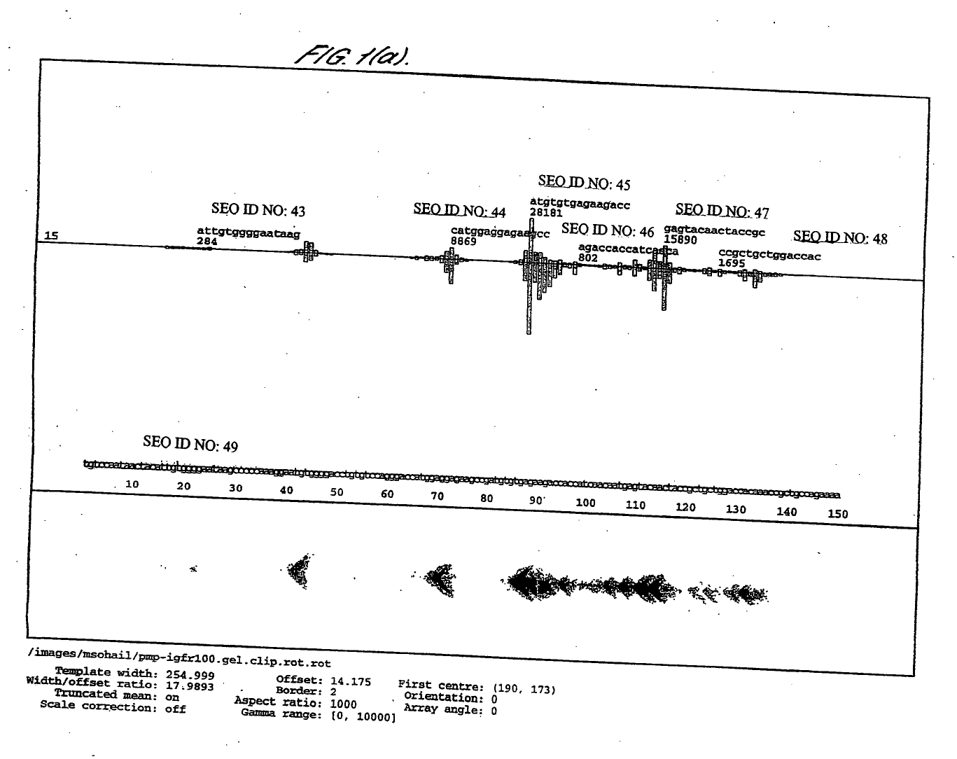

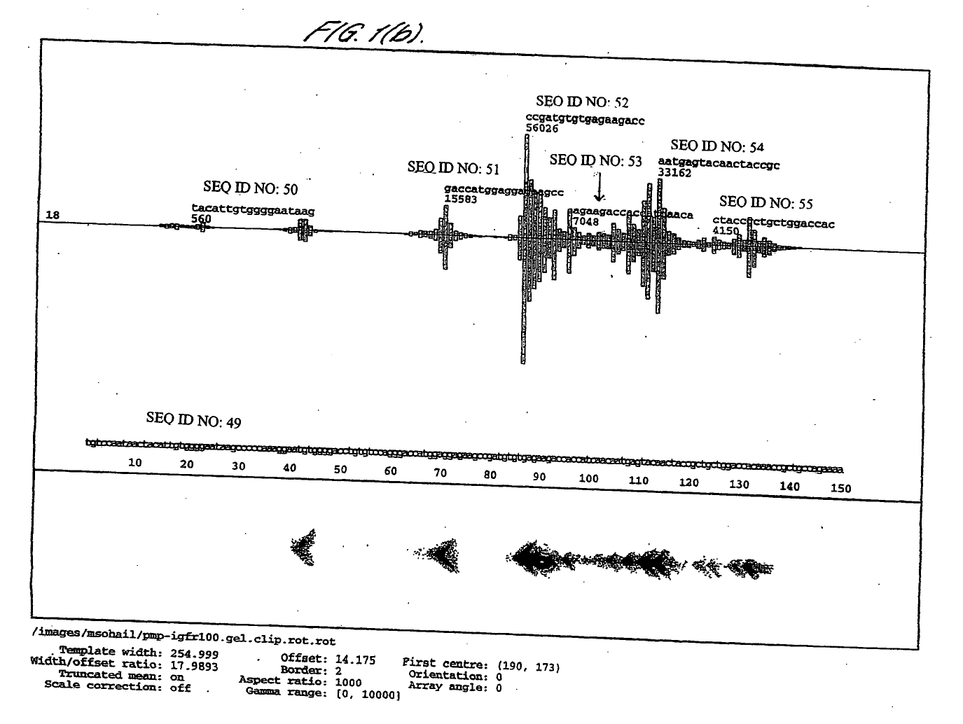

FIG. 1 shows representative plots of hybridisation intensity across the array for (a) 15mers and (b) 18mers probed with labelled IGF1R mRNA.

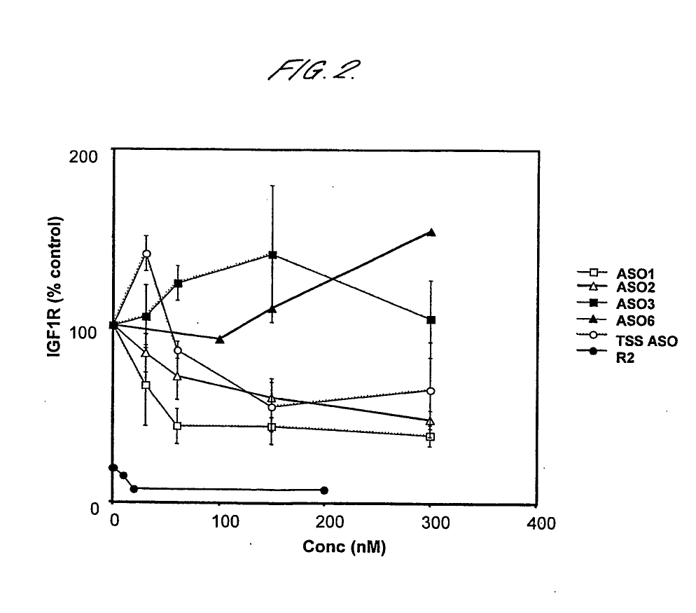

FIG. 2 illustrates the effect of ASCs and RNAi on IGF1R levels in MDA-231 human breast cancer cells. The cells were transfected at 30-40% confluence with phosphorothioate ASOs complexed with the lipid Cytofectin (Glen GSV). After 48 hr the cells were lysed and equivalent amounts of soluble protein were separated by SDS-PAGE and immunoblotted for IGF1R and β-tubulin (loading control). The intensity of the autoradiographic bands was quantified by densitometry, and IGF1R levels were corrected for loading differences. The results are shown as % IGF1R level of that in cells transfected with the same concentration of an appropriate control. This was a scrambled control oligonucleotide for ASOs, and an inverted RNA duplex for RNAi.

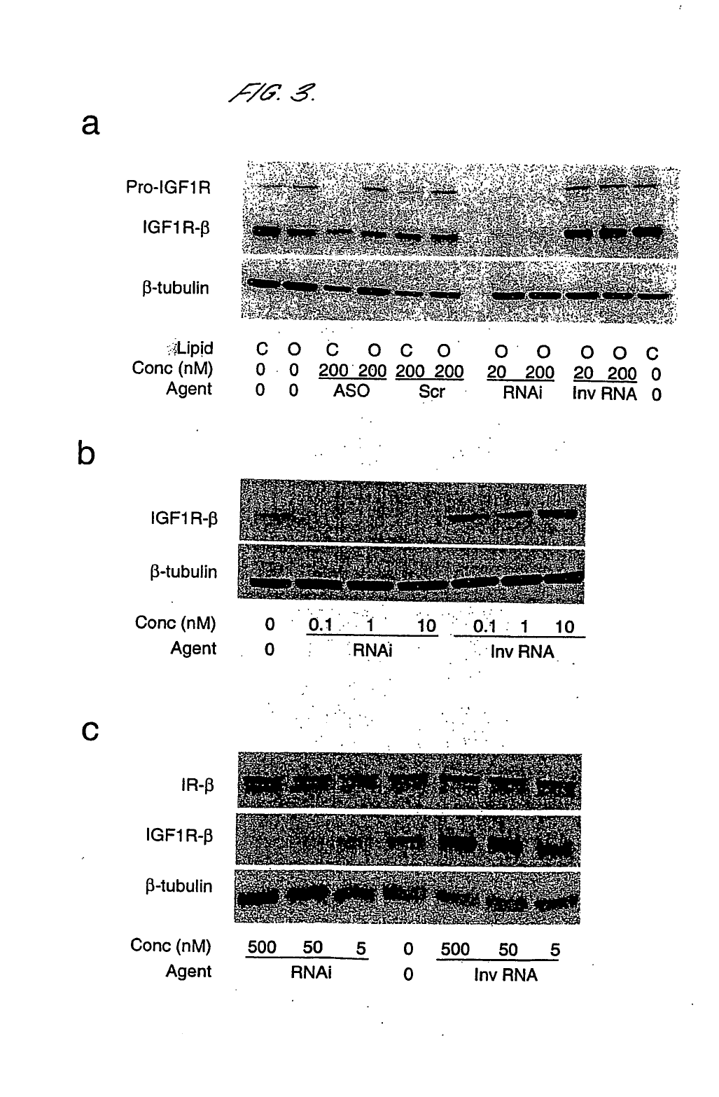

FIG. 3 illustrates the effect of ASOs and RNAi on IGF1R and IR levels in MDA-231 human breast cancer cells and ME melanoma cells. Cells were transfected using either Cytofectin (C) or Oligofectamine (O; Gibco BRL). After 48 hr the cells were lysed and IGF1R and IR levels were determined by immunoblotting.

a) MDA-231 breast cancer cells transfected with ASO 2 (or scrambled control, Scr) or RNAi or inverted RNA duplex control (InvRNA) at 20 or 200 nM.

b) MDA-231 cells were transfected with Oligofectamine and RNAi or Inverted control (InvRNA) at 0.1-10 nM.

c) ME melanoma cells were transfected with Oligofectamine and RNAi or Inv control duplex at 5-500 nM.

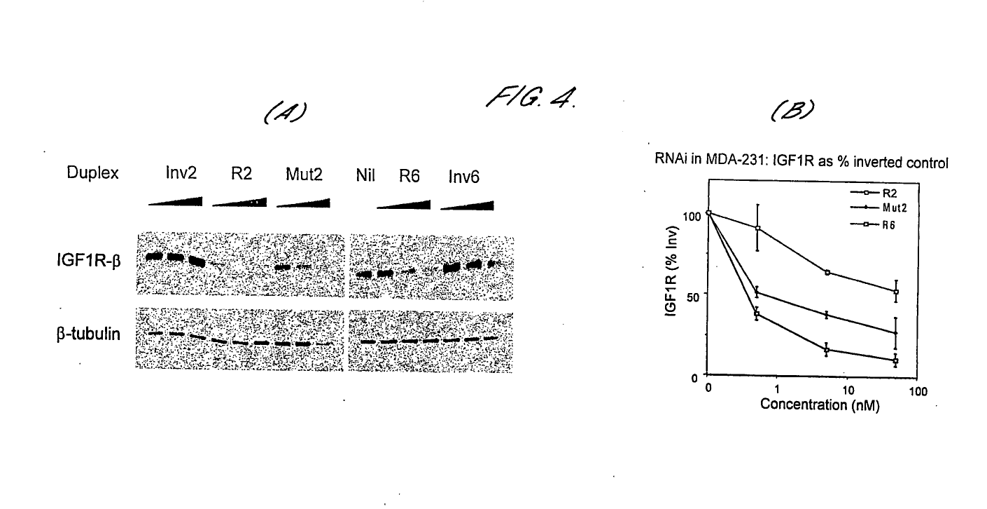

FIG. 4 illustrates the effects of various RNAi duplexes in MDA-231 breast cancer cells. MDA-231 cells were transfected with oligofectamine and 21mer RNA duplexes at 0.5, 5 and 50 nm. After 48 hr IGF1R expression was analysed by immunoblotting (panel (a)). Panel (b) is a graphical illustration, results are presented as % IGF1R level of that in cells transfected with the same concentration of an inverted control RNAi.

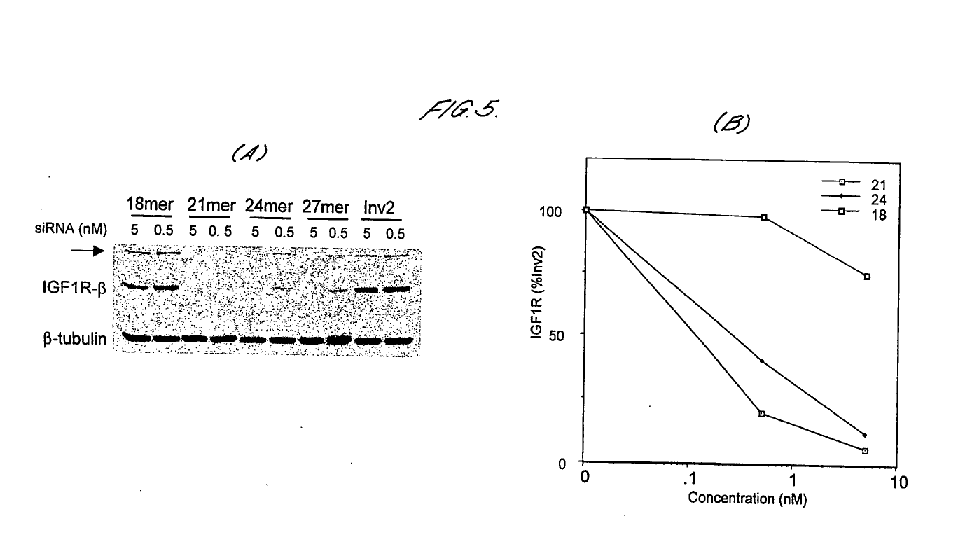

FIG. 5 illustrates the activity of RNAi duplexes of 18, 21 or 24 nt in MDA-231 breast cancer cells. Panel (a) is an immunoblot showing the effect of various RNAis on IGF1R expression at 0.5 and 5 nM; panel (b) is a graphical illustration showing the effect of RNAis of varying length, results are presented as % IGF1R level of that in cells transfected with the same concentration of an Inv2 control RNAi.

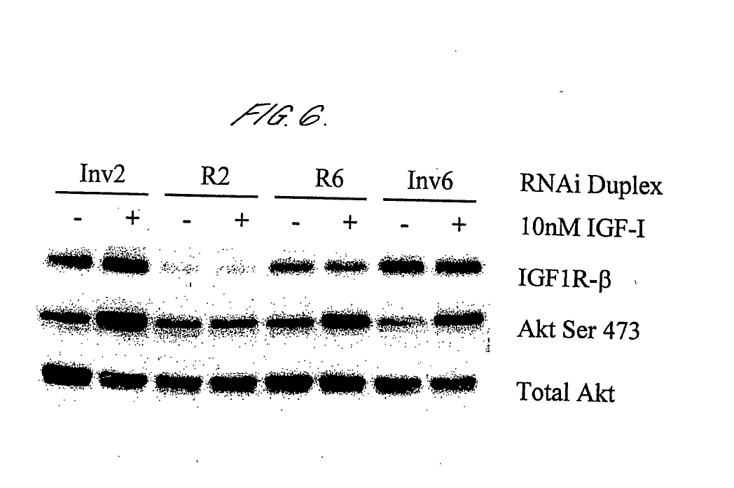

FIG. 6 illustrates the effect of RNAi duplexes on Akt phosphorylation in ME melanoma cells. ME cells were transfected with 100 nM RNA duplexes. After 48 hr the monolayers were disaggregated and half of each culture was treated with 10 nM IGF-1 for 30 min. The cells were lysed and lysates were analysed by immunoblotting for IGF1R, phospho-Ser473-Akt and total Akt.

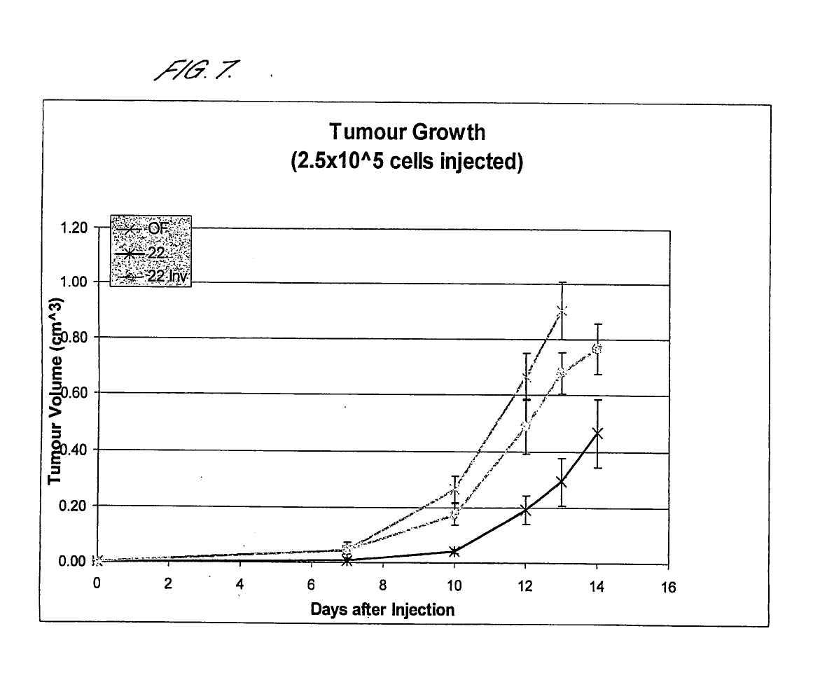

FIG. 7 illustrates growth in vivo in C57BL mice of B16 melanoma cells transfected with RNA duplexes. B16 melanoma cells were transfected with 200 nM duplexes (RNA22=R2,22Inv=Inv2) or oligofectamine alone (OF) for three consecutive days. On the 4th day cells were injected into the flanks of C57BL mice, using 2.5×10⁵ cells/mouse, using groups of 5 mice. Tumours were measured in 2 dimensions every 2-3 days. Tumour volumes were calculated as n(a×b²)/6 where a is the larger and b the smaller dimension.

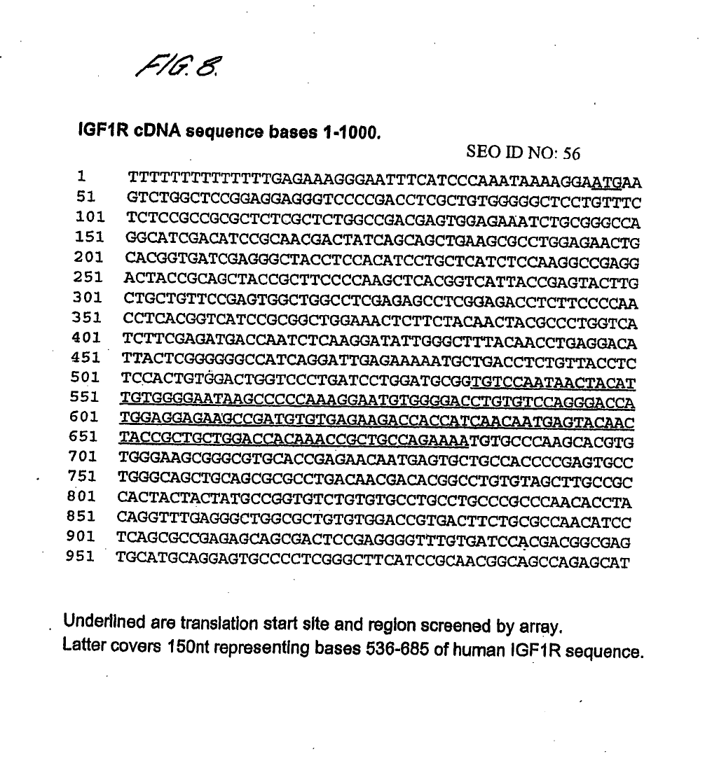

FIG. 8 shows the complete cDNA sequence for human IGF1R. The region evaluated using the scanning array is underlined.

### EXAMPLE 1—SYNTHESIS OF A SCANNING ARRAY

In the following protocol references to Figures and Tables from “Sohail and Southern” refer to Sohail, M. and Southern, E. M. “Using oligonucleotide scanning arrays to find effective antisense reagents”, Methods in Molecular Biology, vol. 170: DNA Arrays: Methods and Protocols, Ed J. B. Rampal, Humana Press Inc., Totowa, N.J.

Using a diamond-shaped or a circular reaction mask (Sohail and Southern, FIG. 1), it is possible to create arrays comprising sets of oligonucleotides of all lengths from monomers up to a maximum in a single series of couplings. The maximum length of oligonucleotides synthesised depends upon the ratio of the diagonal (for a diamond-shaped mask) or diameter (for a circular mask) of the mask to the displacement at each coupling step. For example, a diamond-shaped mask of 40 mm diagonal will produce 10-mers, 16-mers, or 20-mers using step sizes of 4 mm, 2.5 mm, or 2 mm, respectively. A diamond-shaped template creates a series of small diamond-shaped cells. The longest oligonucleotides are found along the centre line and the monomers are located at the edge (Sohail and Southern, FIG. 1). A circular template creates cells that differ in shape: along the centre line, they are lenticular, but off this line, they form a four-cornered “spearhead” that diminishes in size towards the edge. The arrays as synthesised are symmetrical above and below the centre line of the template and each oligonucleotide is represented twice allowing for duplicate hybridisation measurements.

For each length of oligonucleotides s, there are N−s+1 s-mers covering a total length of N bases. For example, if a 150 nt long sequence is covered in a 150 step synthesis, there will be 150 monomers and 131 20-mers. The last 20 positions in the sequence will be represented by shorter oligonucleotides only; in this case, from 19-mer to monomer. Therefore, for making 200 20-mers, an additional 19 nt synthesis steps need to be added at the end, i.e., total coupling steps N+s−1.

Materials

Derivatisation of Glass

1. Glass cylinder and apparatus shown in Sohail and Southern, FIG. 3.

2. Glass sheets of required dimension (3 mm thick: Pilkington, UK).

3. 3-Glycidoxypropyl trimethyoxysilane (98% v/v: Aldrich).

4. Di-isopropylethylamine (99.5% v/v: Aldrich).

5. Xylene (AnalaR: Merck).

6. Hexaethylene glycol (97% v/v: Aldrich).

7. Sulfuric acid (AnalaR: Merck).

8. Ethanol (AnalaR: Merck).

9. Ether (AnalaR: Merck).

10. Water bath at 80° C.

Making Reaction Masks

1. Stainless steel or aluminium square metal piece or PTFE (Teflon). Dimensions of the workpiece may vary according to the size of the mask.

2. A centre lathe or a horizontal milling machine.

3. A drilling machine.

4. Abrasive paper from ˜P600 to P1200 (3M Inc., USA) and polishing grade crocus paper (J. G. Naylor & Co. Ltd., Woodley, Stockport, Manchester, England).

Making Scanning Arrays

1. Solid support (derivatised glass or aminated polypropylene (Beckman Coulter, Inc., USA)).

2. DNA synthesiser (ABI).

3. A reaction mask of desired shape and size and assembly frame (see Sohail and Southern FIG. 4 for the assembly).

4. DNA synthesis reagents: standard dA, dG, dC and T phosphoramidites, oxidizing agent, acetonitrile, activator solution, deblock solution (all from Cruachem).

5. Reverse phosphoramidites bought from Glen Research

Deprotection of Arrays

1. Assembly for constructing deprotection bomb as shown in Sohail and Southern FIG. 5. The assembly consists of a high density polyethylene (HDPE) chamber, 4 mm thick silicon rubber gasket and a stainless steel plate of the dimensions of the HDPE chamber, and stainless steel M8 nuts and bolts.

2. 30% ammonia solution (AnalaR: Merck).

3. Water bath at 55° C.

In Vitro Transcription

1. Template DNA (at ˜1 mg/mL).

2. T7 or SP6 RNA polymerase, transcription buffer (5× transcription buffer is, 200 mM Tris-HCl pH 7.9, 30 mM MgCl₂, 10 mM spermidine, 50 mM NaCl), recombinant RNAsin®, 100 mM DTT and nuclease-free distilled water (Promega).

3. [α-³²P] UTP (3000 Ci/mmol) or [α-³³P] UTP (2500 Ci/mmol) (Amersham).

4. rNTPs (Pharmacia): ATP, GTP, CTP stored as 10 mM solution, and UTP as 250 mM solution in nuclease-free distilled water. Store all reagents at −20° C.

Quantitation of Transcripts

1. Scintillation counter (e.g., Beckman LS 1710).

2. Scintillation Vials and Scintillation Fluid (Amersham).

Hybridisation, Imaging and Analysis

1. Hybridisation buffer (1M NaCl, 10 mM Tris-HCl pH 7.4, 1 mM EDTA, 0.01% SDS (w/v)) (see Note. 2).

2. 50-100 fmol radiolabelled transcript.

3. A glass plate of the size of the array when using an array made on glass, a moist chamber (a large plastic or glass lidded box containing wetted paper towels) and an incubator set to the desired temperature.

4. A hybridisation tube and oven used in standard Southern hybridisation (e.g., Techne) when using an array made on polypropylene.

5. Esco rubber tubing of OD 1 mm (Sterlin) for use in Section 3.8.

6. Storage phosphor screen (Fuji or Kodak).

7. PhosphorImager or STORM (Molecular Dynamics).

8. A SUN Solaris work station for image analysis and the computer software xvseq (L. Wand and J. K. Elder, unpublished) (available by anonymous ftp at ftp://bioch.ox.ac.uk/pub/xvseq.tar.gz).

Stripping of Arrays

1. Stripping solution (100 mM sodium carbonate/bicarbonate buffer pH 10, 0.01% SDS (w/v)) (see Note 3).

2. Geiger-Müller counter (Mini-Instruments Ltd.).

Methods

Derivatisation of Glass

1. Prepare a mixture of di-isopropylethylamine, glycidoxypropyl trimethoxysilane and xylene (1:17.8:69, v/v/v) in a glass cylinder and completely immerse the glass plates in the mixture. Incubate as shown in Sohail and Southern FIG. 3 at 80° C. for 9 h.

2. Remove the plates, allow them to cool to room temperature, and wash with ethanol and then with ether by squirting the liquid from a wash bottle.

3. Incubate the plates in hexaethylene glycol containing a catalytic amount of sulfuric acid (˜25 mL/L) at 80° C. for 10 h, with stirring.

4. Remove the plates, allow them to cool to room temperature, and wash with ethanol and ether. Air dry and store at −20° C.

Machining of Masks

1. Both stainless steel or aluminium can be used to make diamond-shaped and circular reaction masks.

Circular masks are made using a centre lathe and diamond-shaped masks using a horizontal milling machine (see Note 4).

2. To make a diamond shaped mask from metal, hold the work piece at an angle of 45° to the axis of the bed of the milling machine (the diagonal of the diamond running parallel to the axis of the bed.

3. Machine the cavity to the required depth (generally between 0.5-0.75 mm) to create a reaction chamber. Machine the outer lands to a depth of approximately 0.5 mm to form the sealing edge (0.3-0.5 mm wide) (Sohail and Southern FIG. 4).

4. Using the smallest possible diameter cutter (˜1.5 mm), radius the internal corners of the reaction chamber.

5. Finish the sealing edge by polishing flat with successively finer grades of wetted abrasive paper (from ˜P600 to P1200) and finally with a polishing grade crocus paper.

6. Drill holes of 1.08 mm diameter for reagent inlet and outlet, respectively, at the bottom and the top of the reaction chamber (in the corners of the diamonds). Inlet and outlet connections to the DNA synthesiser are made using standard 19SWG syringe needles (1.1 mm diameter) with chamfered ends ground off and de-burred (see Note 5).

Fabrication of Arrays

1. Cut glass or polypropylene to the correct size. The process of making an array is the same when using either glass or polypropylene. Polypropylene has to be mounted on a glass plate, e.g., 3 mm thick soda glass (see Note 6). The total area covered by an array for N bases using a mask of diagonal or diameter D mm and step size 1 mm is N×1+D mm. 2-3 mm are added to margins to allow easy manipulations.

2. Fix the assembly (Sohail and Southern FIG. 4) to the front of a DNA synthesiser and connect its inlet and outlet to the synthesiser's reagent supply.

3. Program the DNA synthesiser with an appropriate synthesis cycle. A slightly modified cycle is used, for example, the one given in Table 1 (Sohail and Southern). Also check all the reagent bottles.

4. Enter the sequence (antisense strand) in 5′ to 3′ direction.

5. Mark the first footprint of the reaction mask on the support by placing it against the mask on the assembly in the desired starting position (see Sohail and Southern FIG. 6). A knife is used to make notches in polypropylene. A diamond scriber can be used to mark glass.

6. Tighten the plate against the mask with the pressure clamp to produce a seal (Sohail and Southern FIG. 4b). Sufficient pressure is applied to stop leakage (˜500-800 Newton force) but not enough to create indentations in the polypropylene surface which can lead to leakage of reagent from the mask during subsequent synthesis steps.

7. Start the DNA synthesiser to go through the pre-programmed cycle to couple the appropriate nucleotide. The first condensation on the substrate is of base at the 3′ end of the sequence.

8. After completion of the step during the interrupt (see Sohail and Southern Table 1 and Note 7), slacken the pressure clamp and move the plate one increment (Sohail and Southern FIG. 4b).

9. Tighten the pressure clamp and start the synthesiser for the next nucleotide in the sequence. Continue the process until the full sequence length is synthesised (see Note 8).

Deprotection of Arrays

1. Place the glass or polypropylene array(s) into the HDPE chamber (see Note 9) and add 30% ammonia into the chamber to cover the array(s) completely.

2. Place the silicon rubber gasket around the rim of the chamber and the stainless steel plate on top of the gasket.

3. Place bolts through the metal plate, the gasket and the HDPE chamber, and tighten.

4. Incubate in a water bath at 55° C. for 12-18 h in a fume hood.

5. Cool the assembly to 4° C. before opening. The arrays are ready to be used in hybridisation at this stage.

Preparing and Quantifying Radiolabelled Transcripts

1. Set an in vitro transcription reaction (20 μL) by adding the following components to a microfuge tube at room temperature.

| | | |
|---|---|---|
| 5× transcription buffer | 4 | μL |
| 100 mM DTT | 2 | μL |
| RNAsin ™ | 20 | U |
| 10 mM ATP, GTP, and CTP | 1 | μL each |
| 250 mM UTP | 1 | μL |
| Template DNA | 2-3 | μL (see Note 10) |
| [a-32P]UTP or [a-33P]UTP | 2 | μL |
| T7 or SP6 RNA polymerase | 20 | U |
| Total Volume | 20 | μL |

Mix and incubate at 37° C. for 1 h.

2. Remove 1 μL for quantitation (see below).
3. Remove unincorporated label by Sephadex® G25 column chromatography (see Note 11).
4. Save 1 μL of the purified transcript for quantitation (see below).
5. Check the integrity of the transcript by denaturing polyacrylamide gel electrophoresis (10).
6. Add 10 μL of the scintillation fluid to the samples saved in Steps 2 and 4.
7. Mix by vortexing and count the samples in a scintillation counter for 1 min.
8. Calculate the per cent-incorporation:

$$\% incorporation = \frac{incorporated cpm \times 100}{total cpm}$$

9. Calculate the amount of RNA made:

$$Amount of [ α - {}^{32} P ] UTP = \frac{20 µCi}{3000 µ Ci \text{/} nmol} = 6.6 \times {10}^{- 3} nmol$$

$$Amount of cold UTP = 1 µL \times 250 µM = 0.250 nmol$$

$$Total UTP = 6.6 \times {10}^{- 3} + 0.25 = 0.256 nmol$$

For a reaction with 50% incorporation, the amount of UTP incorporated
=0.256 nmol÷2=0.128 nmol
Supposing equal incorporation of all four nucleotides, total nucleotides incorporated
=0.128 nmol×4=0.512 nmol
Amount of full-length transcript
=0.512÷ total length of transcript
Hybridisation to Arrays Made on Polypropylene
1. Place the array in the hybridisation tube, coiling it in a spiral.
2. Dilute the radiolabelled transcript in an appropriate volume (10-20 mL depending upon the size of the array and the hybridisation tube) of hybridisation buffer. The mix should cover the array along the length of the tube.
3. Place items 1 and 2 in the oven at desired temperature for 30 min. Also put approximately 100 mL of the hybridisation buffer in the oven: this is to be used to wash the array at the end of hybridisation.
4. Pour the hybridisation mix into the tube containing the array and hybridise for 3-4 h.
5. Remove the hybridisation mix. Briefly wash the array with the hybridisation buffer from Step 3, air dry, cover with cling film and expose to a storage phosphor screen for 16-20 h (see Note 12).
6. Scan the screen on PhosphorImager or STORM and analyse the image using xvseq (see below).
Hybridisation to Arrays Made on Glass
1. Clean the non-array glass plate with acetone and ethanol to ensure it is grease-free and siliconise it by treatment with dimethyl dichlorosilane solution and place it in lidded box. Also place moist paper towel in the box.
2. Dilute the radiolabelled transcript in an appropriate volume of the hybridisation buffer (for example, for an array of 250 mm×50 mm use 500-750 μL).
3. Place items 1 and 2 in an incubator at desired temperature for 30 min. Also put approximately 100 mL of the hybridisation buffer in the oven: this is to be used to wash the array at the end of hybridisation.
4. Using a micropipette, pipette the hybridisation mix in a line evenly along the length of the non-array glass plate, avoiding formation of air bubbles.
5. Starting at one end, carefully place the scanning array (face down) on top of the hybridisation mix. The mix will spread out and form a thin film between the two plates. Incubate for 3-4 h.
6. Separate the plates from each other and wash the array plate with hybridisation buffer to remove unbound mix. Drain the plate, air dry, cover with cling film and expose to a storage phosphor screen for 16-20 h.
7. Scan the screen on PhosphorImager or STORM and analyse the image using xvseq (see below).
Alternative Hybridisation Protocol for Glass or Polypropylene
1. Assemble with clips the array plate (or polypropylene array pasted with PhotoMount™ on a glass plate) and the non-array plate, using rubber tubing as spacers on two sides.
2. Dilute the Radiolabelled Transcript in approximately 5-10 mL of hybridisation buffer.
3. Follow Step 3, Protocol 3.7.
4. Inject the hybridisation mix into the space between the two plates with a needle and syringe.
5. Incubate the assembly in horizontal position at desired temperature.
6. Follow Steps 6 and 7, Protocol 3.7.
Image Analysis

The hybridisation images are analysed using xvseq (see Sohail and Southern FIG. 7). This program reads and displays images generated by a PhosphorImager or STORM and can also perform standard image manipulation such as scaling, clipping and rotation. Although visual inspection of an image reveals the results generally, computer-aided analysis is needed to obtain quantitative information about hybridisation intensities and the oligonucleotide sequences that generated them. xvseq calculates and displays integrated intensities of the array oligonucleotides, each of which corresponds to an image cell formed by intersection of overlapping array templates.

The user can specify the template size, shape and location, step size between successive templates, as well as the sequence that was used to make the array. The template grid is superimposed on the image and the template parameters are adjusted interactively to achieve correct and accurate registration of the grid with the hybridisation pattern. It can be difficult to achieve precise registration by reference to the hybridisation pattern alone, especially, if the signals at either edge of the array are weak or undetectable. Avoid placing the template grid so that it appears to be registered but is in fact misaligned by one or more template steps. Registration can be aided by the use of fixed reference points on an array such as those shown in Sohail and Southern FIG. 6.

Stripping of Arrays

1. The arrays can be used several times. To strip, heat an appropriate volume of the stripping solution to 90° C. in a glass beaker.

2. Immerse the array in the hot stripping solution and stir for 1-2 min.

3. Remove the array and monitor with a Geiger counter to confirm that most of the radiolabel has been removed. Repeat steps 1-2 if radioactivity on the surface of the array is still detectable.

4. Allow the array to cool down to room temperature and wash it thoroughly with nuclease-free distilled water, 70% (v/v) ethanol and finally with absolute ethanol.

5. Air dry and store the array at −20° C. until future use.

Notes

1. The choice of array substrate material and attachment chemistry is important for making high quality arrays. A flat, impermeable surface is required for in situ synthesis of arrays. Glass has a number of favourable qualities, including its wide availability, smooth surface, transparency, chemical stability and compatibility with the use of both radiolabelled or fluorescence labelled nucleic acids targets. Glass is chemically derivatised as described in the methods section to produce a hexaethylene glycol linker which has a terminal —OH group that allows condensation of nucleotide phosphoramidites (Maskos, U., and Southern, E. M. (1992) Nucleic Acids Res. 20, 1679-1684.). Similarly, polypropylene also has favourable physical and chemical properties. Polypropylene is aminated to produce amine groups (Matson, R. S., Rampal, J. B., and Coassin, P. J. (1994) Anal. Biochem. 217, 306-310.) that also allow synthesis to oligonucleotides using standard CE nucleotide phosphoramidites.

For the array fabrication method described here, it is important that a tight seal is formed between the substrate material and the reaction mask. Metals form tight seal with polypropylene but not with glass. PTFE seals well against both glass and polypropylene.

2. 1M NaCl is used routinely. Alternative buffers are: (i) 1 M NaCl, 5-10 mM MgCl₂, 10 mM Tris-HCl pH 7.4, 1 mM EDTA, 0.01% SDS (w/v), and (ii) 150 mM NaCl, 10 mM MgCl₂, 10 mM Tris-HCl pH 7.4, 1 mM EDTA, 0.01% SDS (w/v).

3. Addition of more than 0.01% SDS can damage arrays.

4. Circular masks can also be made from PTFE (Teflon). Diamond-shaped masks are more difficult to make with PTFE by the machining process but can be made by pressure moulding in a hydraulic press (˜150 ton force) using a pre-machined die.

5. Holes should be made as close as reasonably possible to the sealing edge without damaging it. Care must be taken to de-burr fully the holes at the point of entry into the reaction chamber. For PTFE masks the holes should be 1.0 mm diameter which make virtually 100% leak-tight seal. In the case of metal masks, the 0.02 mm interface indicated above also provides a leak-tight seal without the use of any additional sealer. Care must be taken not to insert the end of the syringe needle into the reaction chamber void.

6. Unlike glass, polypropylene is not rigid and thus needs to be mounted on a solid, flat surface for its precise movement against the reaction mask during synthesis. Even mounting of polypropylene on glass is important to produce a good seal between the sealing edge of the reaction mask and the polypropylene surface. Glass used must be clean and free from dust particles because they can cause bulging of the polypropylene which can hinder the formation of a proper seal. A very thin layer of PhotoMount™ (3M Inc, USA) which can be used to paste polypropylene to glass, should be sprayed on glass and not polypropylene.

7. At the start of each synthesis cycle, an interrupt step can be introduced to halt the process at the first step of the next nucleotide condensation cycle to allow the operator to move the plate and restart the program. Alternatively, a long wait step at the beginning of the program can be introduced (see Sohail and Southern Table 1) if the operator does not wish to use the interrupt step. The operator is also advised to consult the user's manual for the DNA synthesiser.

8. With the use of standard phosphoramidites in the synthesis, the oligonucleotides are attached to the solid support at their 3′ ends. Reverse phosphoramidites can be used to make oligonucleotides that are attached at their 5′ ends.

Iodine is used as an oxidising agent. At lower temperatures it will take longer to reach the top of the reaction cell. Iodine can also be replaced with sulfurising agent (Cruachem) to make arrays of phosphorothioate oligonucleotides.

9. When using the standard phosphoramidites, the exocyclic amines of the bases are protected chemically to prevent side reactions during synthesis. These protecting groups need to be removed from the coupled bases before hybridisation.

Before deprotection, detach the polypropylene arrays from glass by peeling from one end. PhotoMount can be removed with ethanol, acetone or dichloromethane.

10. An internally radiolabelled RNA is used as target to hybridise to a scanning array which is generated by in vitro transcription, carried out in the presence of [α-³²P]UTP or [α-³³P] UTP (or [α-³²P] CTP) using an appropriate DNA template. A plasmid containing the desired DNA fragment under the transcriptional control of a T7 or SP6 promoter (such as pGEM: Promega) can be used as template. The plasmid is linearised with an appropriate restriction endonuclease to produce transcripts of defined length without contaminating vector sequence. Alternatively, a template with T7 or SP6 RNA promoter can also be generated using the polymerase chain reaction: primers are used to amplify the required fragment from a plasmid, genomic DNA or cDNA, such that the sense primer has a T7 or SP6 promoter leader sequence (Sohail and Southern Table 2) added at the 5′ end.

11. Sephadex® G25 columns are available from several commercial suppliers including Promega and Pharmacia. Spin columns made in-house, as described in (Sambrook, J., Fritsch, E. F., and Maniatis, T. (1989) Molecular Cloning: A Laboratory Manual. Cold Spring Harbor, N.Y.), can also be used.

12. For hybridisation below 37° C., care must be taken not to touch the plates because this can lead to melting of short duplexes. For hybridisation below room temperature, the cling film and the phosphor screen must be cooled to hybridisation temperature and exposed at the same temperature.

### EXAMPLE 2—USE OF SCANNING ARRAY TO SELECT RNAI REAGENTS

The following is give by way of example of the utility of scanning arrays in the selection of RNAi reagents. The invention is of general utility in the selection of RNAi reagents, thus it is not in any way intended to limit the scope of the invention to this specific example.

Scanning arrays complementary to the region of the IGF1R mRNA from position 537-685 were prepared using the standard techniques described above. The maximum length of the oligonucleotides in the array was either 18 or 20 nt.

In this study hybridisation to the arrays was carried out at physiological temperature (37° C.) in addition to room temperature (23° C.), in order to select sequences which are more likely to have activity in intact cells. The arrays were probed with labelled IGF1R mRNA, and also with labelled insulin receptor mRNA in order to identify oligonucleotides which have high specificity for IGF1R mRNA.

IGF1R Template/Probe Preparation

Human IGF1R cDNA in plasmid pCVN was a generous gift from Renato Baserga. The 5′ region of IGF1R cDNA was cloned into vector pBluescript KS-(Stratagene) using restriction sites HindIII (pCVN-derived site at 5′ end of IGF1R cDNA) and Asp718 (cuts IGF1R cDNA at position 1581). This construct (template 1, 1.6 kb) included approximately 100 bp of polylinker sequence between the T7 promoter and the start of the IGF1R sequence. It was thought that this extraneous vector-derived sequence might influence folding of the transcript. Therefore this region was shortened by restriction digest using Not1, filling in with Klenow enzyme, digestion with EcoRV and re-ligating to make construct 2 (1.6 kbΔ55). Both constructs were linearised at the 3′ end of the IGF1R insert using Asp718, to make templates for in vitro transcription. End-labelled transcripts 1 (1.6 kb) and 2 (1.6 kbΔ55) (see FIG. 1) were generated using T7 RNA polymerase and γ-³²P-GTP (Amersham Pharmacia).

Results

Representative plots of hybridisation intensity across the array for 15mers and 18mers probed with labelled IGF1R mRNA are shown in FIG. 1. The plots are annotated to show the position of selected individual oligonucleotide sequences. In each case the sequence given corresponds to the sense strand of the IGF1R mRNA.

Relative hybridisation intensities for selected oligonucleotides were calculated relative to the most strongly hybridising sequence.

TABLE 1

Relative hybridisation intensity

| | | | |
|---|---|---|---|
| | | Hyb | Rel hyb |
| Oligo | Sequence | intensity | intensity |

| | | | |
|---|---|---|---|
| ASO2 | ggtcttctcacacatcgg | 56,026 | 1.00 |
| ASO4 | gcggtagttgtactcattgt | >33,162 | >0.59 |
| ASO1 | ggcttctcctccatggtc | 15583 | 0.28 |
| ASO3 | attgttgatggtggtcttct | >7048 | >0.13 |
| ASO5 | gtggtccagcagcggtagtt | >4150 | >0.07 |
| ASO6 | cttattccccacaatgta | 560 | 0.01 |

### EXAMPLE 2—SYNTHESIS OF RNAI DUPLEXES

Double-stranded RNA duplexes for RNAi corresponding in sequence to certain of the ASOs identified on the basis of the array screening as hybridising strongly or weakly with IGF1R mRNA were synthesised, together with corresponding inverted controls, mouse homologs and mutant variants (see Table 2). RNA oligonucleotides were synthesised and HPLC purified at Cruachem, Glasgow. Lyophilised oligoribonucleotides were reconstituted in nuclease-free water and diluted to 50 μM. Complementary strands were annealed in 100 mM potassium acetate, 30 mM Hepes-KOH pH 7.4, 2 mM magnesium acetate, as described (Elbashir et al., Nature, 2001, www.dharmacon.com) to give a final concentration of 20 μM duplex. Duplex formation was checked by electrophoresis through 5% low-melting point agarose (NuSieve GTG, BioWhittaker Molecular Applications) in 1xTBE.

TABLE 2
RNAi duplex sequences

| | | |
|---|---|---|
| R2 | GCCGAUGUGUGAGAAGACCTT | SEQ ID NO: 23 |
| | TTCGGCUACACACUCUUCUGG | |
| R2 | GCCGAUGUGUGACAAGTT | SEQ ID NO: 24 |
| (18mer) | TTCGGCUACACACUCUUC | |
| R2 | GCCGAUGUGUGAGAAGACCACCTT | SEQ ID NO: 25 |
| (24mer) | TTCGGCUACACACUCUUCUGGUGG | |
| R2 | GCCGAUGUGUGAGAAGACCACCAUCTT | SEQ ID NO: 26 |
| (27mer) | TTCGGCUACACACUCUUCUGGUGGUAG | |
| Inv2 | CCAGAAGAGUGUGUAGCCGTT | SEQ ID NO: 27 |
| | TTGGUCUUCUCACACAUCGGC | |
| Mut2 | GCCGAUGUGUGUGAAGACCTT | SEQ ID NO: 28 |
| | TTCGGCUACACACACUUCUGG | |
| MouseR2 | GCCCAUGUGUGAGAAGACCTT | SEQ ID NO: 29 |
| | TTCGGGUACACACUCUUCUGG | |
| R6 | CUACAUUGUGGGGAAUAAGTT | SEQ ID NO: 30 |
| | TTGAUGUAACACCCCUUAUUC | |
| Inv6 | GAAUAAGGGGUGUUACAUCTT | SEQ ID NO: 31 |
| | TTCUUAUUCCCCACAAUGUAG | |
| MouseR6 | CUACAUUGUGGGGAACAAGTT | SEQ ID NO: 32 |
| | TTGAUGUAACACCCCUUGUUC | |

For comparison purposes the following antisense oligonucleotides were also synthesised:

TABLE 3
Antisense oligonucleotides

| | | |
|---|---|---|
| ASO1 (18mer) | GGCTTCTCCTCCATGGTC | SEQ ID NO: 1 |
| ASO1 (20mer) | CGGCTTCTCCTCCATGGTCC | SEQ ID NO: 2 |
| Mouse ASO1 | GGGCTTCTCCTCCAATGTCC | SEQ ID NO: 3 |
| Scr1 | TCTTCCGCGACTTGCTCCGC | SEQ ID NO: 4 |
| Mouse Scr1 | CTGTTCACCGTGCACCCTGT | SEQ ID NO: 5 |
| ASO2 (18mer) | GGTCTTCTCACACATCGG | SEQ ID NO: 6 |
| ASO2 (20mer) | TGGTCTTCTCACACATCGGC | SEQ ID NO: 7 |
| Mouse ASO2 | TGGTCTTCTCACACATGGGC | SEQ ID NO: 8 |
| Scr2 | CCTGTACGCGTTGATCTCCA | SEQ ID NO: 9 |
| Mouse Scr2 | AGTCGCCTAGTCGAGTCCTT | SEQ ID NO: 10 |
| ASO3 | ATTGTTGATGGTGGTCTTCT | SEQ ID NO: 11 |
| Scr3 | TCATGCTTGTTGATGTGGTT | SEQ ID NO: 12 |
| ASO4 | GCGGTAGTTGTACTCATTGT | SEQ ID NO: 13 |
| Scr4 | TGTCGTTCGAGTTGATCGTA | SEQ ID NO: 14 |
| ASO5 | GTGGTCCAGCAGCGGTAGTT | SEQ ID NO: 15 |
| Scr5 | GACGTTAGCGTGCGATGTGC | SEQ ID NO: 16 |
| ASO6 | CTTATTCCCCACAATGTA | SEQ ID NO: 17 |
| Mouse ASO6 | CTTGTTCCCCACAATGTA | SEQ ID NO: 18 |
| Scr6 | CATAGCACATTCTTCTCA | SEQ ID NO: 19 |
| Mouse Scr6 | ACCGTTCATCAGTTCACT | SEQ ID NO: 20 |
| TSS | TCCTCCGGAGCCAGACTT | SEQ ID NO: 21 |
| ScrTSS | CAGCTACTCGCATGCTGC | SEQ ID NO: 22 |

### EXAMPLE 3—TESTING OF RNAI DUPLEXES

Cell Culture and Transfection

The human and murine cell lines used in this study were cultured in RPMI 1640 plus 10% FCS at 37° C. in a humidified atmosphere of 5% CO₂. The cultures were negative when tested for Mycoplasma infection. Cultures were passaged the day before transfection to achieve 30-50% confluence the following day. Initial transfections of ASOs used 1-2.5 μg/ml Cytofectin (Glen Research, Sterling, Va.), according to the manufacturer's instructions. Latterly Oligofectamine (Life Sciences) has been used for all ASO and RNAi transfections. The method was as described (see manufacturer's instructions and www.dharmacon.com) with minor modifications. For transfection of monolayers seeded in 6 well plates, 5 μl phosphorothioate oligonucleotide or RNA duplex at 100× final concentration were mixed with 250 μl serum-free Optimem (Gibco-BRL). In a separate tube 2 μl Oligofectamine was mixed with 68 μl Optimem. After 10 min incubation at room temperature the contents of the two tubes were mixed and incubated for a further 25 min at room temperature. Monolayers were washed with 1-2 ml Optimem. To the cells were added 175 μl Optimem followed by the 325 μl complexes. Volumes were scaled up by factors of 2.22 or 6.05 for transfection in 60 mm or 100 mm dishes respectively. Cultures were incubated at 37° C. for 4 hr, and then 50 μl FCS and 3.5 ml RPMI plus 5% FCS were added to each well. After 48 hr incubation effects on IGF1R and IR expression were determined by immunoblotting. Some transfected cultures were disaggregated using 3 mM EDTA in PBS for 2-4 min, washed in serum-free RPMI, divided into two aliquots and treated with 10 nM Long R3—IGF-I (GroPep, Adelaide) or diluent for 30 min at 37° C. prior to lysis.

Immunoblotting

IGF1R expression was assessed by immunoblotting as previously described (Macaulay et al. 2001). After washing in ice-cold PBS, cells were lysed in 50 mM Hepes pH 7.5, 100 mM NaCl, 10 mM EDTA, 1% Triton-X-100, 4 mM sodium pyrophosphate, 2 mM sodium orthovanadate, 10 mM sodium fluoride, 1 mM PMSF, 2 μg/ml each leupeptin and aprotinin. Lysates were centrifuged for 15 minutes at 14,000 g, the protein concentration of supernatants was measured using BCA assay reagent (Pierce), and equivalent amounts of protein were separated on 7.5% SDS-PAGE gels and transferred to nitrocellulose. Target protein levels were assessed using antibodies to the β-subunit of the IGF1R or IR (Santa Cruz), phospho-Ser 473 Akt or total Akt (Cell Signalling, New England Biolabs) or β-tubulin (Sigma). Primary antibodies were detected with HRP-conjugated secondary antibodies (Dako), and ECL Plus (Amersham Pharmacia).

Results

(i) Down-regulation of IGF1R in MDA-231 cells by antisense oligonucleotides and RNAi:—comparison of the activity of ASOs and RNAi.

ASOs and RNAi duplexes were transfected into MDA-231 cells using Cytofectin or Oligofectamine, as described above. After 48 hr the cells were lysed, and IGF1R and IR levels were measured by immunoblotting. The intensity of the autoradiographic bands was quantified by densitometry, and IGF1R (or IR) levels were corrected for loading differences. The specific IGF1R or IR results are presented as % IGF1R (or IR) level of that in cells transfected with the same concentration of an appropriate control. This was a scrambled control oligonucleotide for ASOs, and an inverted RNA duplex for RNAi. The results are shown in Table 4 and FIG. 2.

TABLE 4
Effect of IGF1R-ASOs on IGF1R and
IR expression in MDA-231 cells

| Protein Level (% control) | |
|---|---|
| IGF1R | IR |

| ASO | 30 nM | 300 nM | 30 nM | 300 nM |
|---|---|---|---|---|
| 1 | 66 ± 23 | 38 ± 6 | 112 | 135 |
| 2 | 84 ± 11 | 47 ± 5 | nd | nd |
| 3 | 105 ± 18 | 104 ± 22 | nd | nd |
| 4 | 73 ± 2 | 49 ± 2 | 97 | 92 |
| 6 | nd | 100 | nd | 100 |
| TSS | 141 ± 10 | 64 ± 19 | 144 | 59 |

The effects of an ASO and RNAi duplex of equivalent sequence (ASO2 and R2) on IGF1R and IR expression were compared in MDA-231 human breast cancer cells and ME melanoma cells. The transfection protocol was as above, using either Cytofectin (C) or Oligofectamine (O; Gibco BRL). After 48 hr the cells were lysed and IGF1R and IR levels were determined by immunoblotting. Representative immunoblots are shown in FIG. 3.

a) MDA-231 breast cancer cells transfected with ASO 2 (or scrambled control, Scr) or RNAi (sequence R2 given above) or inverted RNA duplex control (InvRNA) at 20 or 200 nM.

b) MDA-231 cells were transfected with Oligofectamine and RNAi or Inverted control (InvRNA) at 0.1-10 nM.

c) ME melanoma cells were transfected with Oligofectamine and RNAi or Inv control duplex at 5-500 nM.

Quantitative analysis of these results is incorporated into the graph of ASO effects in MDA-231 (FIG. 2).

It is clear that RNAi causes much more profound inhibition of IGF1R expression than occurs with ASOs. Note that the quantification methods may not be linear at very low protein levels, and it is possible that these analyses underestimate the true extent of IGF1R downregulation. For example the immunoblots (FIG. 5) show almost complete inhibition of IGF1R expression (even on these overnight exposures) in MDA-231 breast cancer and ME melanoma cells treated with 10-200 nM RNAi.

A second RNAi (R6 in FIG. 4; sequence R6 in Table 2) was synthesised corresponding to ASO 6, which hybridizes to IGF1R mRNA on the array with a relative intensity of 0.01. While this does cause downregulation of the IGF1R, the effects were significantly less potent than that of the RNAi corresponding to ASO2 (R2 in FIG. 4; sequence R2 in Table 2). RNAi R2 is more effective than RNAi R6 in the following cell lines:

| | |
|---|---|
| DU145 | prostate |
| A549 | NSCLC |
| UC101 | ovary |
| U20S | osteosarcoma |
| MCF7 | breast (ER positive) |

This result indicates that intensity of hybridisation to the scanning oligonucleotide array is predictive of the activity of RNAi reagents.

The inhibitory effect of RNAi R2 is partially, though not completely, blocked by the presence of a single base pair mutation (sequence Mut2, in FIG. 4), see FIG. 4, compare R2 with Mut2. The effect of R6 was less than the effect of the mutant duplex Mut2. This indicates that the efficacy of synthetic 21mer RNAi molecules is influenced by secondary structure in, and hence access to, the target region of the mRNA.

(iv) Comparison of RNAi Duplexes R2 and R6 in a Range of Human and Murine Cell Lines

Tumour cells were transfected with 10 nM duplexes and IGF1R levels were measured after 48 hr. After correction for loading differences IGF1R levels in cells transfected with RNAi were expressed as % of levels in cells transfected with equivalent inverted control duplex.

TABLE 4
Effect of R2 and R6 RNAi duplexes
in human and murine cell lines

| Cell line | Cell type | R2 | R6 |
|---|---|---|---|
| DU145 | Human prostate cancer | 34 ± 13 | 74 ± 8 |
| A549 | Human non-small cell lung | 22 ± 2 | 102 ± 16 |
| ME | Human melanoma | 53 ± 3 | 104 ± 5 |
| B16 | Murine melanoma | 45 ± 4 | 72 ± 14 |

In some cell lines eg human ME melanoma and A549 NSCLC, 10 nM R6 was essentially inactive. In all cases R2 caused more profound IGF1R downregulation than R6. This suggests that the structural features dictating access are robust and conserved between different cell lines and species.

(v) Effect of RNAi Duplexes of Varying Length

Compared with the effects of R2, IGF1R downregulation is less profound after treatment with an 18mer duplex (sequence as R2 but lacking the 3 RNA bases at the 3′ end). In contrast to the results of Elbashir et al. (2001), a 24mer R2 duplex was observed to be as effective as R2 at 100 nM. Results are illustrated in FIG. 5. Panel (a) is an immunoblot showing the effect of various RNAis on IGF1R expression at 0.5 and 5 nM; panel (b) is a graphical illustration showing the effect of RNAis of varying length, results are presented as % IGF1R level of that in cells transfected with the same concentration of an Inv2 control RNAi. A 27mer R2 duplex was also observed to be as effective as the R220mer and 24mer duplexes (data not shown).

### EXAMPLE 4—DIFFERENTIAL EFFECTS OF RNAI DUPLEXES ON IGF SIGNALING

ME cells were transfected with 100 nM RNA duplexes. After 48 hr the monolayers were disaggregated and half of each culture was treated with 10 nM IGF-1 for 30 min. The cells were lysed and lysates were analysed by immunoblotting for IGF1R, phospho-Ser473-Akt and total Akt (see FIG. 6).

The IGF1R downregulation that follows transfection with R2 was sufficient to inhibit IGF-1-induced Akt phosphorylation, while R6 caused modest IGF1R downregulation that was not sufficient to block IGF-I signalling to Akt (FIG. 6). This is significant because it indicates that R2, which corresponds to an ASO hybridising strongly to the scanning array, not only caused more potent IGF1R downregulation but that this translated to a significant biological advantage; Akt phosphorylation is the major anti-apoptosis pathway downstream of the IGF1R.

### EXAMPLE 5—GROWTH OF CELLS TRANSFECTED WITH RNAI DUPLEXES IN VIVO

B16 cells were transfected with 200 nM RNA duplexes (RNA22=R2, 22Inv=Inv2) or oligofectamine alone (OF) for three consecutive days. On the 4th day cells were injected into the flanks of C57BL mice, using 2.5×10⁵ cells/mouse, using groups of 5 mice. Tumours were measured in 2 dimensions every 2-3 days. Tumour volumes were calculated as π(a×b²)/6 where a is the larger and b the smaller dimension.

B16 cells transfected with R2 showed reduced growth rate in vivo, (syngeneic C57BL mice) compared with cells transfected with inverted control RNA duplex (FIG. 7). This results indicates that an RNAi reagent selected on the basis of hybridisation to a scanning array is effective in vivo.

1. A method of preparing an siRNA reagent for use in gene silencing of a target gene by RNA interference, which method comprises:
(a) preparing a scanning array of antisense oligonucleotides spanning a region of a transcript of the target gene;
(b) hybridising to the array labelled transcripts of the target gene;
(c) identifying an oligonucleotide within the array which hybridizes with the labelled transcripts; and
(d) preparing an siRNA reagent comprising a double-stranded RNA of identical sequence to the oligonucleotide identified in step (c).

2. A method of preparing a pharmaceutical composition comprising an siRNA reagent capable of mediating gene silencing of a target gene by RNA interference, which method comprises:
preparing an siRNA reagent capable of mediating gene silencing of a target gene by RNA interference according to the method of claim 1 and formulating the siRNA reagent into a pharmaceutical composition comprising the siRNA reagent and one or more diluents, excipients or carriers.

3. A method of preparing an expression vector capable of expressing an siRNA reagent for use in gene silencing of a target gene by RNA interference, which method comprises:
(a) preparing a scanning array of antisense oligonucleotides spanning a region of a transcript of the target gene;
(b) hybridising to the array labelled transcripts of the target gene;
(c) identifying an oligonucleotide within the array which hybridizes with the labelled transcripts; and
(d) preparing an expression vector capable of expressing an siRNA reagent comprising a double-stranded RNA of identical sequence to the oligonucleotide identified in step (c).

4. A method of preparing a pharmaceutical composition comprising an expression vector capable of expressing an siRNA reagent for use in gene silencing of target gene by RNA interference, which method comprises:
preparing an expression vector capable of expressing an siRNA reagent for use in gene silencing of a target gene by RNA interference according to the method of claim 3 and formulating the expression vector into a pharmaceutical composition comprising the expression vector and one or more diluents, excipients or carriers.
